(19) United States
(12) Patent Application Publication
(10) Pub. No.: US 2025/0018084 A1
(43) Pub. Date: Jan. 16, 2025

(54) LIQUID RADIOEMBOLIC AGENTS AND RELATED EMBOLIZATION SYSTEMS AND METHODS OF EMBOLIZATION

(71) Applicant: ICAHN SCHOOL OF MEDICINE AT MOUNT SINAI, New York, NY (US)

(72) Inventors: Alejandro BERENSTEIN, New York, NY (US); Benjamin Isaac RAPOPORT, New York, NY (US); Munir V. GHESANI, New York, NY (US); Joshua B. BEDERSON, New York, NY (US)

(73) Assignee: ICAHN SCHOOL OF MEDICINE AT MOUNT SINAI, New York, NY (US)

(21) Appl. No.: 18/708,774

(22) PCT Filed: Nov. 14, 2022
(86) PCT No.: PCT/US2022/049793

§ 371 Date: May 9, 2024

Related U.S. Application Data
(60) Provisional application No. 63/278,697, filed on Nov. 12, 2021.

(51) Int. Cl. A61L 24/02; A61L 24/00; A61L 24/06

(52) U.S. Cl. CPC A61L 24/02; A61L 24/0015; A61L 24/06; A61L 2300/418; A61L 2300/44; A61L 2400/06; A61L 2430/36

(57) ABSTRACT

A kit is provided for delivering local radiation to a tumor while also causing tumor devascularization. The kit includes a first liquid radioembolic agent for treatment of at least a first region of the tumor and a second liquid radioembolic agent for treatment of at least a second region of the tumor. The first liquid radioembolic agent includes: a biocompatible polymer or prepolymer and a first radioisotope having a first type of ionizing radiation for treatment of the first region of the tumor. The second liquid radioembolic agent includes: a biocompatible polymer or prepolymer and a second radioisotope having a second type of ionizing radiation for treatment of the second region of the tumor. The second type of ionizing radiation is different than the first type of ionizing radiation. Each of the first type of ionizing radiation and the second type of ionizing radiation is selected from the group consisting of: alpha type ionizing radiation, beta type ionizing radiation: gamma type ionizing radiation; and combinations thereof.

### CROSS REFERENCE TO RELATED APPLICATION

The present application claims priority to and the benefit of U.S. patent application Ser. No. 63/278,697, filed Nov. 12, 2021, which is hereby incorporated by reference in its entirety.

### TECHNICAL FIELD

The present disclosure relates to liquid radioembolic agents and an embolization system (kit) that includes two or more liquid radioembolic agents for delivery to and treatment of target tissue, such as a tumor (e.g., a meningioma) or vascular malformation. Methods for assessing dosimetry and efficacy of embolization are also disclosed.

### BACKGROUND

The body's vascular system includes arteries and veins. Arteries carry high-oxygen blood away from the heart towards the rest of the body. Arteries branch out into many smaller arteries in other parts of the body. As blood travels through the arteries, it loses oxygen. Veins carry the blood back to the heart to absorb more oxygen.

As is known, the purpose of embolization is to prevent blood flow to an area of the body, which can effectively shrink a tumor or block an aneurysm, commonly carried out as an endovascular procedure. Vascular embolization can be used to prevent or control bleeding (e.g., organ bleeding, gastrointestinal bleeding, blood vessel bleeding, bleeding associated with aneurysms) or can be used to block blood supply as in the case of excising a tumor. Endovascular embolization of blood vessels is a surgical treatment option for a variety of purposes, including endovascular treatment of tumors and treatment of injuries such as aneurysms, arteriovenous malformations, and arteriovenous.

Tumors (e.g., solid mass tumors) require arterial blood flow and therefore, all of the tumor's blood supply comes from one or more arteries. A solid tumor is an organ composed of neoplastic cells and host stromal cells nourished by the vasculature made of endothelial cells-all embedded in an extracellular matrix. Embolization techniques are thus equipped to take advantage of this by placing a catheter into the arteries feeding the tumor and then delivering an embolic agent to cause vascular embolization of blood vessels feeding the tumor to induce necrosis of the tumor tissue by obstructing its arterial supply.

Over the past century, embolization has grown as a minimally invasive technique to achieve vascular occlusion. Several classes of embolization agents (embolic agents) are commercially available, including mechanical (coils or plugs), particles/gelatin, and liquid/gel-based embolics. The choice of agent is dependent on the clinical context, vessel size, durability (temporary vs permanent), and operator preference.

Liquid embolic agents are used across many different therapeutic applications and thus have widespread use. One of the properties specific to liquid embolic agents is their ability to fill the target vessel and induce vascular occlusion by advancing with blood flow and penetrating deeper into the vascular bed to areas where a catheter or coil may not reach. The precise mechanism by which the occlusion occurs varies depending on the type of liquid embolic utilized. Currently, liquid embolization is conducted using two different type of embolic agents, some adhesive and some non-adhesive; and using different classes of catheters: non-detachable catheters, detachable tip microcatheters, and balloon microcatheters.

Some embolic agents have a short lived action such as collagen and gelfoam, whereas others, like glue or coils, are permanent. Liquid embolic agents include, but are not limited to, Onyx™, alcohol, ALGEL, and Phil™, N-Butyl Cyanoacrylate (nBCA, glue) has also been utilized as a permanent embolic agent. nBCA is diluted with ethiodol and tantalum. nBCA displays a fast polymerization rate when exposed to the ionic environment of blood. Ethiodol is used as vehicle and a polymerization retardant. Onyx™, a mixture of ethylene alcohol vinyl polymer (EVOH), dimethyl sulfoxide (DMSO) and tantalum powder for radiopaque visualization, has been approved by the FDA for embolization of cerebral AVMs.

The solvent for Onyx™ embolization is dimethyl sulfoxide (DMSO). DMSO prevents Onyx premature hardening in the catheter. During the embolization procedure the clinician injects DMSO when the catheter is in position. Consequently, Onyx is injected, moving the column of DMSO towards the distal catheter tip. When Onyx comes in contact with blood, DMSO diffuses away and the hardening process begins. In contrast with nBCA, Onyx™ requires DMSO compatible catheters. nBCA is an adhesive agent and Onyx™ is cohesive and non-adhesive, acting like lava and displaying progressive solidification and cohesiveness hardening from the inside out. Importantly, due to its cohesive nature, Onyx allows for slower injection times.

As mentioned above, traditionally, microcatheters are used to deliver an embolic agent to a target site (e.g., a vascular site). The type of microcatheter that is used depends on a number of factors including the type of embolic agent being used and the type of therapeutic treatment being pursued. Embolization of tumors is usually performed using microcatheters for different reasons. At first, there is a requirement for localized embolization for effecting primarily the tumor and as little healthy tissue as possible. A microcatheter is usually passed via a larger-lumen catheter, which is placed within the proximal part of the vessel, such as the celiac or hepatic artery, and the microcatheter is then advanced therethrough towards the tumor until reaching an effective distance for the embolization. It is often advantageous to use a diagnostic catheter as the delivery medium for the microcatheter, by not replacing it with a larger diameter sheath, for example, therefore saving substantial time.

There is substantial literature and experience in radio—and chemoembolization for oncologic applications outside the central nervous system, in other areas of interventional radiology and interventional oncology. Cancers of the liver have received particular attention, and Yitrium-90 microspheres are currently in use. However, these techniques have not been widely applied in the central nervous system (CNS).

For example, chemoembolization is a palliative treatment of liver cancer. This can be a cancer originating in the liver or a cancer that has spread (metastasized) to the liver from other areas of the body. During chemoembolization, three chemotherapy drugs are injected into the artery that supplies blood to the tumor in the liver. The artery is then block off (embolized) with a mixture of oil and tiny particles. Chemoembolization advantageously accomplishes at least the following: (1) the tumor becomes deprived of oxygen and nutrients once the blood supply is blocked; (2) because the drugs are injected directly at the tumor site, the dosage can be 20 to 200 times greater than that achieved with standard chemotherapy injected into a vein in the arm; (3) because the artery is blocked, no blood washes through the tumor and as a result, the drugs stay in the tumor for a much longer time; and (4) there is a decrease in side effects because the drugs are trapped in the liver instead of circulating throughout the body. It will therefore be appreciated that the embolization aspect of chemoembolization involves the use traditional embolic agents and traditional embolization techniques.

Endovascular embolization as an adjunct to surgical treatment of meningiomas and other head and neck tumors is used to decrease vascularity in order to facilitate surgery. Embolization can also induce necrosis within a tumor, leading to decreased mass effect and symptoms over days to weeks. Surgical treatment of meningiomas has been accompanied by perioperative mortality ranges from 0% to 9.4%. Reported complication rates after radiosurgery range from 2% to 16%, and it may require months or years before radiated tumors respond to treatment. Embolization is also being investigated as a minimally invasive treatment for meningiomas and other head and neck tumors and has been associated with complication rates <1%.

The efficacy of embolization in the brain, head, and neck has been limited by (1) a lack of therapeutic agents designed to directly cause tumor necrosis (as opposed to decreasing blood flow) and (2) a lack of methods to quantify the extent of embolization.

While surgical resection and radiation remain the main contemporary definitive treatments for tumors of the brain, head and neck (most of which respond incompletely to chemotherapy), tumor embolization has proven to be an effective surgical adjunct. Embolization involves catheter-based delivery of substances to the arterial network of the tumor to diminish or eliminate the tumor blood supply. Due to the angiogenic burden induced by many tumors, preoperative embolization has gained traction because of the associated decrease in blood loss and resulting tumor necrosis, which can facilitate bloodless surgery and more complete resection of softer or partially necrotic tumors. In other fields, as in cancer of liver, embolization may provide definitive therapy. Embolization is typically carried out with commercial embolization agents such as microparticles (Hydropearl, for example, manufactured by Terumo Interventional Systems, Somerset, NJ), or liquid embolic agents, such as Onyx (Medtronic, Minneapolis, MN) or n-BCA glue (Trufill, Johnson & Johnson, New Brunswick, NJ), which solidify in specific chemical environments. These agents produce various degrees of mechanical occlusion, which can produce various degrees of ischemia, but no biological effect to the embolized tissue/territory.

The efficacy of embolization in the brain, head, and neck has been limited by two factors: (1) poor ability to quantify extent of embolization; and (2) lack of therapeutic agents designed not only to stop blood flow to the tumor, but also to cause tumor necrosis in a direct manner.

### SUMMARY

In one aspect of the present disclosure, an embolization kit is provided for delivering local radiation to a tumor while also causing tumor devascularization. The kit includes a first liquid radioembolic agent for treatment of at least a first region of the tumor and a second liquid radioembolic agent for treatment of at least a second region of the tumor. The first liquid radioembolic agent includes: a biocompatible polymer or prepolymer and a first radioisotope having a first type of ionizing radiation for treatment of the first region of the tumor. The second liquid radioembolic agent includes: a biocompatible polymer or prepolymer and a second radioisotope having a second type of ionizing radiation for treatment of the second region of the tumor. The second type of ionizing radiation is different than the first type of ionizing radiation. Each of the first type of ionizing radiation and the second type of ionizing radiation is selected from the group consisting of: alpha type ionizing radiation, beta type ionizing radiation; gamma type ionizing radiation; and combinations thereof.

In another aspect of the present disclosure, a method is disclosed for embolizing a blood vessel leading to or in a solid mass tumor and causing necrosis to a portion of the solid mass tumor. The method includes the steps of: (1) identifying at least one blood vessel that leads to or is in the solid mass tumor; (2) injecting a first liquid radioembolic agent into the at least one blood vessel, the first liquid radioembolic agent including a biocompatible polymer or prepolymer and a first radioisotope and being injected into the blood vessel under conditions wherein the polymer or prepolymer polymerizes and forms a solid mass which embolizes the at least one blood vessel and further wherein the first radioisotope is employed in an amount effective to cause necrosis of at least a first portion of the tumor; and (3) injecting a second liquid radioembolic agent into the at least one blood vessel, the second liquid radioembolic agent including a biocompatible polymer or prepolymer and a second radioisotope and being injected into the blood vessel under conditions wherein the polymer or prepolymer polymerizes and forms a solid mass which embolizes the at least one blood vessel and further wherein the second radioisotope is employed in an amount effective to cause necrosis of at least a second portion of the tumor.

### BRIEF DESCRIPTION OF THE DRAWINGS

FIG. 1A is a view of an exemplary solid mass tumor (e.g., a meningioma) showing the vascular system that feeds the tumor;

FIG. 1B illustrates a first method of treatment in which alpha type ionizing radiation is delivered to the periphery of the tumor and beta type ionizing radiation is delivered to the core of the tumor;

FIG. 1C illustrates a second method of treatment in which beta type ionizing radiation is delivered to the periphery of the tumor and alpha type ionizing radiation is delivered to the core of the tumor;

FIG. 1D illustrates a third method of treatment in which alpha, beta and gamma type ionizing radiation is delivered to the periphery of the tumor and alpha, beta, and gamma type ionizing radiation is delivered to the core of the tumor;

FIG. 2 is an illustrate of a pre-operative MRI of the head showing the presence of a solid mass tumor (e.g., a sphenoid wing meningioma);

FIG. 3 is an angiogram of the tumor location of FIG. 2 showing tumor blush;

FIG. 4 is an angiogram distinguishing vascular pedicles supplying tumor (and subsegments of tumor) from vascular pedicles supplying adjacent non-tumor tissue;

FIG. 5 is a microcatheter angiogram confirming tumor blush with no delivery of embolic agent to surrounding non-tumor tissue;

FIG. 6 is an enlarged fluoroscopic image confirming the presence of a center embolus and a periphery embolus relative to a tumor; and

FIG. 7 is an enlarged gamma camera image of the tumor of FIG. 6 showing radioactivity in both the center embolus and the periphery embolus.

### DETAILED DESCRIPTION OF CERTAIN EMBODIMENTS

In general, the present disclosure is directed to: (1) an embolization agent (embolic composition) that delivers small amount of short-half-life, local radiation to a tumor while also stopping blood flow to the tumor to induce necrosis; (2) a system and method of embolization using the embolization (embolic) agent; and (3) a computational system for quantifying the degree of embolization in real time, through automated analysis of digital angiograms, obtained during the embolization procedure, using convolutional neural networks.

The present disclosure is thus directed to a system and method for embolizing tumors not only to stop blood flow as an adjunct to therapy (i.e., embolization of blood vessels supplying blood to the tumor), but also as a definitive treatment, by incorporating radioactive agents into the embolic material that directly induce tumor necrosis (as a result of providing therapeutic levels of radiation to the blood vessel and/or surrounding tissue). The embolic agent, including the radioactive agent, (which can be referred to as being a radioembolic agent) is delivered, e.g., to a vascular site, as a fluid and solidifies in vivo to form a solid, coherent mass. This process allows for the treatment of tumors that may otherwise be inoperable.

It will be understood, as described herein, that the radioembolic agents described herein are delivered through the vascular system of the patient. In other words, the radioembolic agents can be delivered through one or more arteries, through one or more veins or through a combination of the two depending upon a number of parameters, such as the location of the tumor, size of the tumor, etc.

While there are benefits to a treatment plan that includes intraarterial delivery of a radioactive agent, there are also associated limitation and deficiencies. For example, the delivery of just a radioactive agent to the tumor can suffer from the following: (1) risk of systemic toxicity, most notably to the thyroid gland and (2) overall, very low therapeutic index. Similarly, while studies show the effectiveness of a treatment plan that is exclusively based on embolization of intracranial neoplasms, there can also be associated limitations and deficiencies. Ischemia induced by embolization decreases tumor size. However, there is a risk of recurrence or progression as the tumor cells in the penumbra may escape ischemic damage. The radioembolic agents described herein and the associated treatments disclosed herein combine the strengths of two proven techniques in a single setting. The bulk of tumor would still respond to ischemia induced by embolization. Radiation provides additional boost by inducing single or double strand DNA damage to the tumor cells already being targeted by ischemia.

Local irradiation by a radioembolic agent is known to serve as a low-dose-rate brachytherapy and is a paradigm that has proven effective in cancers of the liver. HepatiItc radioembolization through the use of ⁹⁰Y has been explored in multiple clinical studies and in particular, studies have confirmed that macrodosimetry is a useful tool for understanding dosage of this radioactive agent, and that radioembolization resulted in tumor necrosis. However, radioembolization has not been reported in neurosurgery studies and therefore, the present disclosure is directed to a novel system and method.

In another aspect, the present disclosure describes a method for embolizing a blood vessel leading to or is in a tumor and causing necrosis to at least a portion of the tumor. After identification of the tumor to be treated using conventional imaging techniques, a treatment plan is formulated and involves, at least in part, identification of tumor feeding vessels that can be utilized in an embolization procedure. This includes the identification of one or more blood vessels which lead to or is in the tumor itself. This involves planning considerations concerning the delivery pathway of the microcatheter and any possible restraints on such delivery of the microcatheter relative to the target location for embolization.

The radioembolic agents described herein have particular utility in treating meningiomas; however, they can equally be used to treat other types of tumors. As is known, a meningioma is a common type of brain tumor that develops slowly in the meninges, or the area that covers and protects the brain and spinal cord. Most meningiomas are benign and can vary greatly in size and location. Brain meningiomas: most meningiomas occur in the brain and include: (1) convexity meningiomas usually grow towards the front of the brain, on its surface. Almost 20 percent of meningiomas fall into this category. One usually does not see any symptoms until the tumor becomes large. At that point, the patient may experience seizures, headaches, and changes in vision, as well as neurological impairment; (2) falcine and parasagittal meningiomas grow between the two sides of the brain, where there are many large blood vessels. This type of tumor can interfere with blood circulation in the brain, if it is sitting on surrounding blood vessels; and (3) intraventricular meningiomas grow within the ventricles of the brain, which carry cerebrospinal fluid. A tumor in this area can block the flow of the fluid and can produce headaches and dizziness. Skull base meningiomas: skull base meningiomas grow under the brain and along the base of skull. These tumors may be more difficult to remove surgically than brain meningiomas because they may be on or near the bones of the skull. Skull base meningiomas include: (1) carvernous sinus meningiomas are rare tumors that affect the cavernous sinus, an area that controls eye movement and allows your face to feel sensations. Cavernous sinus meningiomas can cause double vision, dizziness and facial pain; (2) clival meningiomas are located on the underside of the cerebrum within the posterior cranial fossa. These types of meningiomas often grow as part of a larger lesion within the sphenoid bone; (3) foramen magnum meningiomas start off in the hole in the base of the skull that the spinal cord passes through (called the foramen magnum); (4) olfactory groove meningiomas grow near the olfactory nerve, located between the brain and the nose. If you have an olfactory meningioma, you could lose your sense of smell. If the tumor becomes very large, it can affect your vision; (5) posterior fossa/petrous meningiomas are located on the underside of the brain. They can cause facial pain, such as trigeminal neuralgia, and can produce spasms in the face; (6) sphenoid wing meningiomas form on the sphenoid ridge behind the eyes. These meningiomas can cause visual problems and facial numbness. In severe cases, they can cause blindness. Spinal meningiomas are less common than other types of skull base meningiomas and typically occur in middle-aged women. The tumors press against the spinal cord in the thoracic region of the chest and can cause back pain, numbness, and tingling.

In one embodiment, the radioembolic agent (radioembolic composition) comprises at least: (a) a biocompatible polymer or prepolymer and (b) a radioisotope. The radioisotope can be a water insoluble radioisotope. The amount and radioactive content of the radioisotope is sufficient to provide a therapeutic effect and more particularly, to effect necrosis of at least a portion of the tumor. The radioembolic agents are delivered, for example, directly to the tumor (e.g., solid mass tumor) or to a vascular site selected to be in or near the tumor and the amount and radioactive content of the selected radioisotope is sufficient to affect such necrosis.

A biocompatible polymer is a polymer which, in the amount employed, is non-toxic and substantially non-immunogenic when used internally in the patient and are also substantially insoluble in blood. A biocompatible prepolymer is a polymeric material which polymerizes in situ to form a polymer and which, in the amount employed, is non-toxic and substantially non-immunogenic when used internally in the patient and are also substantially insoluble in blood.

Broadly, the radioembolic agent includes a suitable liquid embolic agent with a suitable radioisotope that can function as a contrast agent to assist in visualization of the formed mass. Alternatively, a non-radioactive contrast agent can also be used in combination with the radioisotope in order to ensure visualization.

As mentioned herein, one common liquid embolic agent is nBCA which is an adhesive type liquid embolic agent. In the present radioembolic compositions, the nBCA comprises the biocompatible (pre) polymer component of the radioembolic composition.

nBCA Liquid Embolic Agent

Suitable intravascular compositions include, by way of example only, cyanoacrylates which polymerize in vivo to form a solid mass as well as solutions of a biocompatible, water insoluble polymer dissolved in a non-aqueous solvent such as dimethyl. sulfoxide (“DMSO”) whereupon introduction into the vasculature, the DMSO dissipates and the polymer precipitates in the aqueous based blood composition. Cyanoacrylate glues are thus suitable for and are used as embolics as well as a tissue adhesive.

A cyanoacrylate glue takes the form of a clear, radiolucent liquid that can be injected via a catheter into the desired vascular tree. The ability of nBCA to travel distally with the flow of blood is advantageous in certain applications in which deep penetration into a vein is required. nBCA polymerizes and solidifies, forming a cast when it contacts ionic fluid (e.g., blood, saline). This results in thrombosis, localized endothelial inflammation that leads to an exothermic reaction that forms byproducts such as formaldehyde, and ultimately, local fibrosis, creating permanent vascular occlusion. As mentioned herein, given the affordability and rapid onset of action, nBCA has many applications, including but not limited to, embolization of AVMs, endoleaks after endovascular aneurysm repair (EVAR), acute hemorrhage, selective portal vein embolization, low-flow venous malformations, chyle leak, lymphatic malformations, and end-organ embolization, such as for renal angiomyolipomas (AMLs).

The rate of nBCA polymerization can be modulated with glacial acetic acid (GAA) or other suitable solutions. The use of GAA thus allows the surgeon to customize the delivery of several different radioembolic agents within the same vascular pedicle, whereby the polymerization rates are controlled to achieve the intended result which may be the delivery in series of one radioembolic agent to one locations and another radioembolic agent to another location. As the amount of GAA within the mixture increases, the rate of polymerization slows.

### Therapeutic Radioactive Contrast Agent

In accordance with the present disclosure, the radioembolic agent can include a therapeutic radioactive contrast agent to not only assist in visualization of the formed mass but also provide therapeutic treatment. In particular, radioisotopes that are suitable for the applications described herein form part of the embolization agents described herein. As is known, radioisotopes are radioactive isotopes of an element. Radioisotopes can also be defined as atoms that contain an unstable combination of neutrons and protons, or excess energy in their nucleus.

Suitable radioisotopes include radioisotopes of iodine. For example, iodine-125 (¹²⁵I) is a radioisotope of iodine which has uses in biological assays, nuclear medicine imaging and in radiation therapy as brachytherapy to treat a number of conditions, including prostate cancer and brain tumors. Iodine-125 has a radioactive decay half-life of about 59.49 days. Iodine-125 is a beta emitter.

Iodine-131 (¹³¹I) is another important radioisotope of iodine that is used in medical applications. Iodine-131 has a radioactive decay half-life of about 8 days. Iodine-131 is a beta and gamma emitter.

Other suitable beta emitter radioisotopes include, but are not limited to: Y-90, Lu-177, and Cu-67.

Suitable alpha emitter radioisotopes include, but are not limited to: Pb-212, Ac-225, Ra-223, and At-211.

It will be appreciated that Iodine based radioisotopes are only exemplary in nature of the types of radioisotopes that can be used and therefore, other alpha, beta and gamma type radioisotopes can be used as long as they are suitable, and are administered in proper amounts, for the intended applications described herein.

### EXAMPLE

In one embodiment, the embolic agent comprises a liquid preparation of radioactive Iodine-125 in combination with n-butyl cyanoacrylate (nBCA). The radioactivity of the embolic agent can be calibrated, and the material can be injected via microcatheter in controlled fashion, is visible in real time during injection under fluoroscopic guidance and polymerizes only in contact with blood plasma. The characteristics of Iodine-125 make it an ideal embolic agent for tumors of the brain, head, neck, and spine. Iodine-125 is a beta-emitter, and so the level of radioactivity is extremely localized, with negligible penetration into the tissue that surrounds the embolized tumor.

The use of liquid radioembolization agents have significant advantages to particulate agents, as it can be delivered through smaller lumen more flexible catheters required in the cerebral circulation, and by delaying the polymerization time, the radioembolization agents penetrate further in the microcirculation. In addition, the liquid radioembolization agents deliver a more uniformed radiation.

In one embodiment, the liquid embolic agent comprises:
(1) Cyanoacrylates, derived from ethyl cyanoacrylates and related esters (including but not limited to n-butyl cyanoacrylate (nBCA), octyl-cyanoacrylate, 2-octyl cyanoacrylate, methyl-2-cyanoacrylate, ethyl-2-cyanoacrylate) tissue adhesives; and
(2) a radioisotope, such as a radioisotope of Iodine (e.g., Iodine-125 or Iodine-131), that functions as a contrast agent to assist in visualization of the formed mass.

In addition, and according to another embodiment, a suitable isotope or agent can be embedded in liquid embolic materials compatible with vascular, interstitial or topical central nervous system (CNS), or other body applications. For example, a fraction of the tantalum can be substituted with a radioactive material in nBCA (or other cyanoacrylates) glues, or Onyx, or other DMSO based liquid embolic formulations. Moreover, other suitable isotopes that can be used include isotopes with alpha and/or beta radiation, that may be suited for different parts of the tumor, for example, at the borders of the tumor adjacent normal tissues. For example, an alpha emitter with a shorter penetration may be best for use at the peripheral borders of the tumor, whereas a beta emitter is likely more effective for the center of the tumor.

### Homogeneity of the Liquid Radioembolic Agent

As is known, the term homogenous means uniform in structure or composition. A homogenous liquid is thus a fluid with uniform properties throughout.

As mentioned herein, in chemoembolization, a mixture includes microparticles is used to block off the artery; however, in such mixture, the microparticles are not homogenously distributed and therefore, after injection, the microparticles likewise are not distributed in a homogenous manner. This is part results from the microparticles being solid and thus, the mixture is not completely liquid but has a solid component. As a result of the non-homogenous distribution of the microparticles, the radiation diffuses throughout the tumor in a non-homogenous way resulting in some areas of the tumor being exposed to greater radiation than other areas which is less than optimal.

In contrast, the radioembolic agents disclosed herein are liquid embolic agents and therefore, the radioisotope is distributed in a homogenous manner throughout the liquid glue (cyanoacrylate) component. As a result, when injected and when cured to a mass at the target location, the radiation diffuses throughout the tumor in a homogenous way resulting in more optimal distribution of radiation throughout the tumor that is not possible with microparticle based embolics.

In one example, Applicant delivered a radioembolic agent that included I-131 as the radioisotope to a target location (target volume). The results showed that the distribution of I-131 was homogenous throughout the target volume. Very long half-life of I-131 (˜8 days), combined with the fixed distribution accomplished by the radioembolization technique results in a powerful tumoricidal effect for an extended period, which would in turn significantly improve patient outcomes.

### Liquid Radioembolic Agents Combining Different Radiation Types

Existing treatments are limited to the administration of single types of radiation (as in proton therapy, for example). In contrast, the present embolic agents and methods of treatment described herein permit the combination of different radiation types. For example, alpha, beta, and/or gamma radiation types can be combined. Alpha versus beta particles have different properties, such as different linear energy transfers. Alpha particles travel very short distances, giving high energy deposition in that short distance. Beta particles, on the other hand, travel longer distance but give away less energy per unit distance.

Accordingly, the liquid radioembolic agents can be formulated with isotopes emitting alpha, beta, gamma, or combinations thereof. The present teachings therefore allow for different types of radiation to be used separately or in combination. In other words, and according to a first embodiment, the liquid radioembolic agent comprises a liquid mixture of two or more different radioisotopes (e.g., an alpha radioisotope and a beta radioisotope).

In addition, as described herein, gamma emitting radioisotopes can be used in certain applications and advantageously, functions as a diagnostic tool.

Alternatively, two more different radioembolic agents can be used in series to treat the tumor using the same vascular pathway (e.g., same vascular pedicle) or using two different vascular pathways (e.g., use of two different vascular pedicles). In one example, the method of embolization can include the use of a first liquid radioembolic agent for the center region of the tumor and use of a second liquid radioembolic agent for the peripheral border region of the tumor. The first liquid radioembolic agent can include a beta emitting radioisotope and the second liquid radioembolic agent can include an alpha emitting radioisotope. The first liquid radioembolic agent can be delivered with a first microcatheter and the second radioembolic agent can be delivered with a separate second microcatheter.

The selection of the radioisotopes and their intended targets will vary depending upon the tumor characteristics and the treatment plan. For example, while in a first treatment plan, a beta radioisotope is delivered to the tumor's center and an alpha radioisotope is delivered to the tumor's periphery, the opposite can be true in that in an alternative plan, an alpha radioisotope can be delivered to the center and beta radioisotope to the periphery.

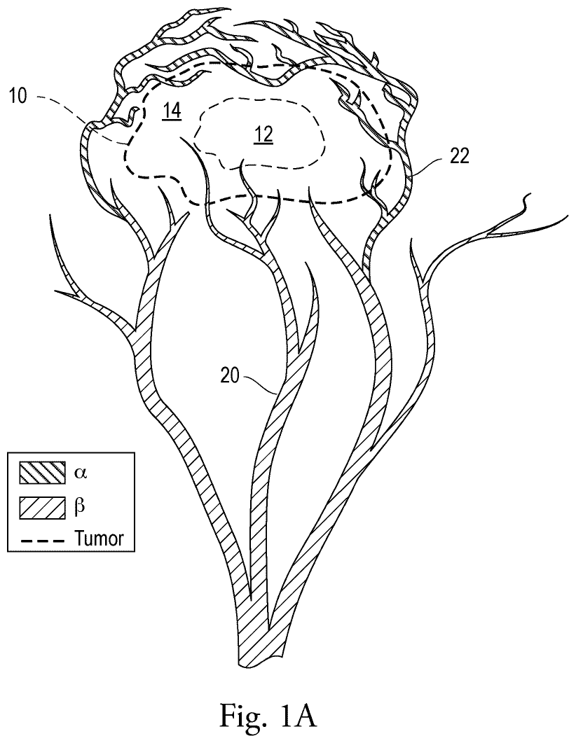

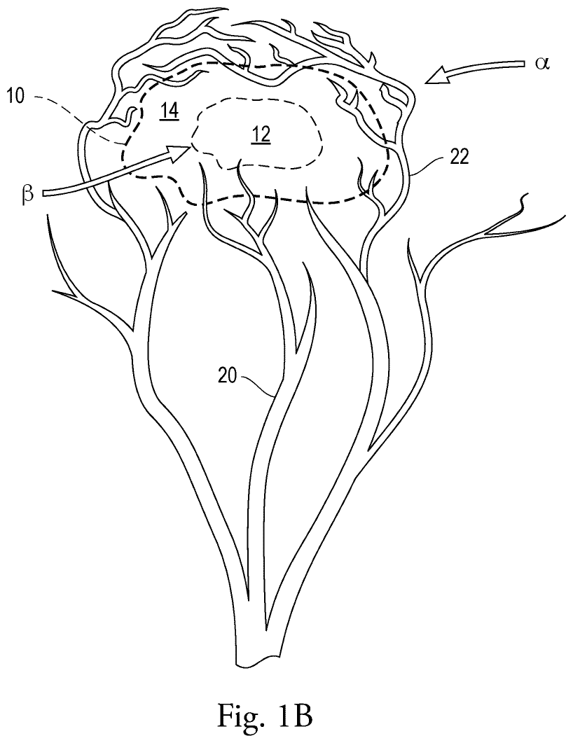

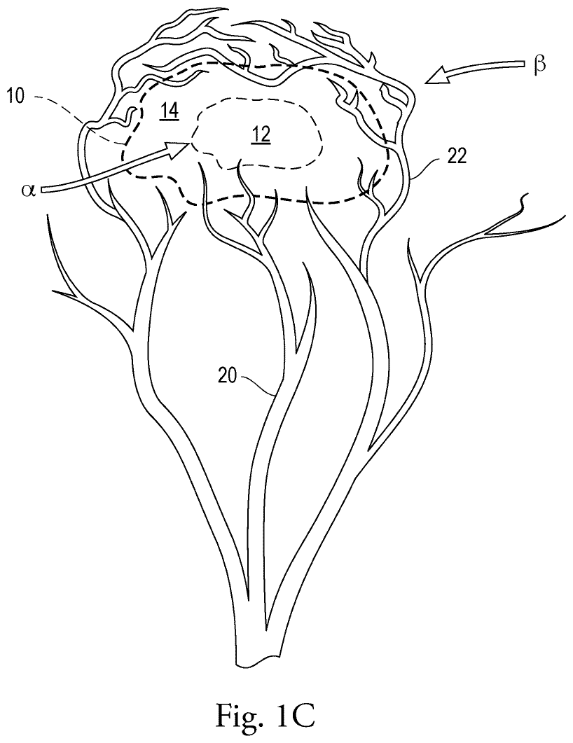

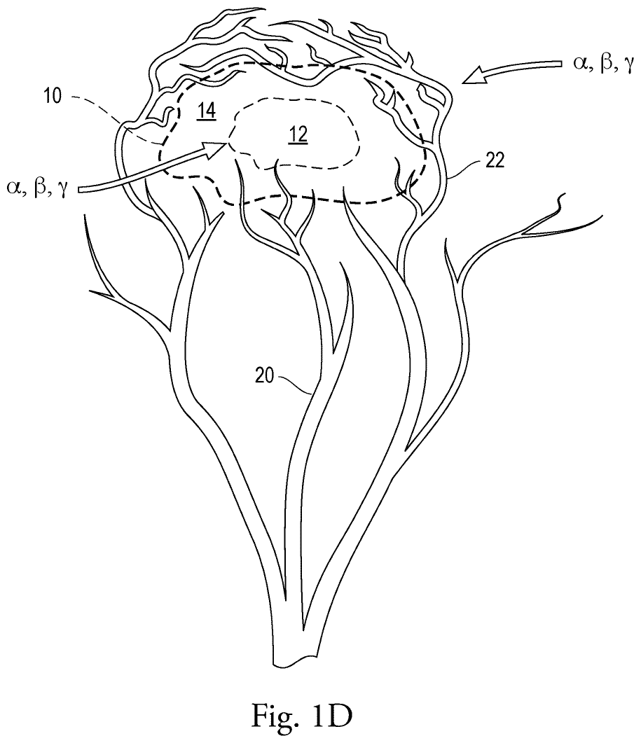

FIG. 1A illustrates an exemplary tumor 10 (e.g., a meningioma). The tumor 10 includes a center region or core 12 and an outer peripheral region 14 that surrounds the center region 12. For purpose of discussion, the center region 12 has a first arterial supply that can be described as being defined by a first vascular pedicle 20, while the outer peripheral region 14 has a second arterial supply that can be described as being defined by a second vascular pedicle 22. One skilled in the art will readily understand that the identification and selection of the arterial pathways for delivering the radioembolic agents depend on a number of factors and considerations, including, but not limited to, the tumor location, the vascular anatomy of the tumor, etc. These consideration will guide the surgeon in generating the optimal surgical/treatment plan. For example, with respect to a convexity meningioma, the dominant tumor vascular supply is from the middle meningeal artery (ECA) which is embolized; while the tumor vascular supply is from ICA branches which are not embolized for safety concerns and therefore, there is an opportunity for the targeted radioembolization described herein. Accordingly, the core of the tumor can be targeted with a beta emitter radioisotope (part of the radioembolic agent), while the periphery is targeted with an alpha emitter radioisotope (part of the radioembolic agent) (FIG. 1B). Alternatively, a first radioisotope mixture that includes both alpha and beta emitter radioisotopes can be delivered to the tumor core and similarly, a second radioisotope mixture that includes both alpha and beta emitter radioisotopes can be delivered to the tumor periphery. FIG. 1C illustrates a treatment plan with alpha type ionizing radiation delivered to the core and beta type ionizing radiation delivered to the periphery.

It will be appreciated that the cross-hatching in FIG. 1A is meant to depict the alpha and beta pathways (vascular pedicle pathways) as opposed to the locations at which the emboli (“casts”) are formed in the vascular pedicle pathways. In other words, the emboli are not formed along the entire cross-hatched areas in FIG. 1A but rather these are the pathways that are targeted for delivering radiation (e.g., alpha or beta radiotherapy) to the identified regions of the tumor (e.g., core or periphery).

For treatment of an orbital roof meningioma, the first pedicle (middle meningeal artery) is used for delivery of a better emitter radioisotope (e.g., by a first radioembolic) to the tumor core and a second pedicle (ophthalmic artery) can be targeted for delivery of an alpha emitter radioisotope (e.g., by a second radioembolic).

In this way, the user can target different areas of tumor for different types of treatment. More specifically, the particular radioisotope that is included in the liquid embolic agent can be chosen in view of the desired depth of penetration of the radiation within the tumor. For example, for beta emitters can be chosen for a greater depth of penetration, while alpha emitters can be chosen for a less depth of penetration. Accordingly, tumor cells in the penumbra escaping damage from ischemia alone are now targeted by beta particles that have sufficient linear energy transfer to penetrate the zone of penumbra, while peripheral areas are targeted by alpha particles. The alpha and beta radiation combination therefore would induce tumor cell damage in both the central and peripheral zones alike.

In other words, one of the advantages of the present radioembolic agents and related embolization system is that, unlike conventional brachytherapy seeds, these embolic agents penetrate deeply into the smallest arteries and capillaries and do not simply form macroscopic plugs in the feeding arteries. This feature enables highly uniform delivery of radiation by the radioisotope(s) to the target tissue.

### Example

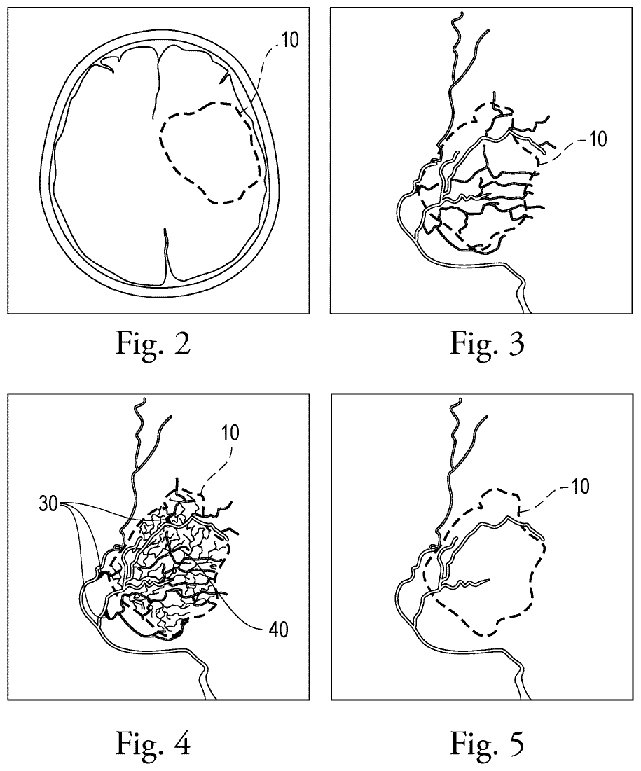

FIGS. 1A-5 illustrate an exemplary treatment plan and use of the radioembolic agents described herein. First, an MRI image confirms the existence of the meningioma 10 as shown in FIG. 2. Next, a diagnostic angiogram is performed as shown in FIG. 3. As is known, an angiogram is a diagnostic procedure that uses imaging to show how a patient's blood flows through the blood vessels or heart. An injected contrast material makes it easy to see where blood is moving and where blockages are. X-rays or other types of imaging are used for the angiogram. FIG. 4 shows embolization via the use of one or more microcatheters that deliver one or more radioembolic agents to the site. In the angiogram image of FIG. 4, tumor blush is visible. Tumor blush is an enhancement of the tumor on radiologic examinations by administration of contrast agents. FIG. 4 illustrates the formation of several emboli 30 that are formed at or near the periphery of the tumor 10, as well as one or more emboli 40 formed at or near the center of the tumor 10.

FIG. 5 is an angiogram image showing devascularization (loss of the blood supply to a bodily part due to destruction or obstruction of blood vessels) and one will notice in FIG. 5 the lack of tumor blush which is indicative of the successful devascularization of the tumor. For ease of illustration the emboli 30, 40 are not shown.

Based on the foregoing, it will be appreciated that the user can prepare a targeted, custom treatment plan for the patient. It will be appreciated that the personalized treatment plan, that depends on the factors below, can be designed to obtain maximum tumoricidal effect while minimizing side effects. These factors, include but are not limited to: (1) size of the lesion: determinant of total radiation dose needed for optimal effect; (2) location of the lesion: in a more sensitive lesion you want to decrease the side effect to the surrounding tissue by using higher proportion of alpha particles at the periphery; and (3) tumor vascularity: less vascularity and larger penumbra would require higher proportion of beta particles at the periphery.

### Example

Tumors are mixtures of different compartments and can be classified as such. In accordance with the present disclosure, the radioembolization technique can be tailored and customized based on tumor compartment structure. For example, in the case of a tumor with four compartments, each compartment can be treated individually with either a radioembolic including an alpha emitter, a radioembolic including a beta emitter, or any combinations thereof.

### Gamma Radiation and Gamma Camera Confirmation

In one aspect, at least one radioisotope used in the embolization method can be a gamma emitter type radioisotope. FIG. 1D shows a method of treatment in which alpha, beta and gamma type ionizing radiation is delivered to the periphery of the tumor and alpha, beta, and gamma type ionizing radiation is delivered to the core of the tumor.

The use of a gamma emitter radioisotope can provide not only a therapeutic effect but also provides a valuable diagnostic tool. In particular, after the embolization procedure is completed, a gamma camera can be used to provide confirmation that the embolus is formed and more particularly, that the embolus has radioactive properties. A gamma camera or SPECT camera is a camera that is able to detect scintillations (flashes of light) produced when gamma rays, resulting from radioactive decay of single photon emitting radioisotopes, interact with a sodium iodide crystal at the front of the camera.

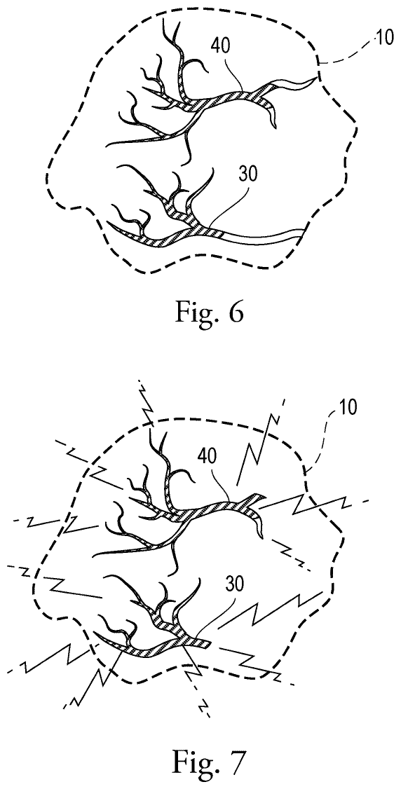

FIG. 7 shows an image from a gamma cameration with the dark regions (emboli 30, 40) indicating areas of active radioactivity (i.e., the location(s) of the formed embolus(es)). Other imaging confirmation techniques, such as fluoroscopy (FIG. 6), can be used to provide confirmation of the formed embolus. Fluoroscopy is based on the presence of a radiopaque material in the formed emboli 30, 40.

The generation of post-operative images, including fluoroscopic images and gamma camera images, provides visual confirmation of both the location of the embolus and more importantly, confirmation of its radioactivity.

Gamma-emission thus permits confirmation of extent of (radiation) penetration of the embolized tumor and the use of a gamma emitter radioisotope allows one to perform subsequent confirmatory imaging and precise computation of dosimetry. The gamma camera can be used in real time to image a distribution of radiation within the target tissue location and more particularly, a plurality of gamma camera images can be taken over a predetermined period of time to determine and observe radiation levels over the predetermined period of time. The plurality of gamma camera images can then be used to perform dosimetric calculations.

More specifically, the use of the gamma radioisotope in the radioembolic agent allows for imaged-based dosimetry to be performed. As is known, in imaged-based dosimetry, a plurality of images (e.g., gamma-camera images) are acquired at select time points during a time period after administration of the radioembolic agent(s). Existing software allows for tumors and organs to be delineated in the images and the amount of activity quantified. According to certain dosimetry methods, timeactivity curves can be obtained from the planar images for the respective tissue and can be used to calculate the absorbed dose for any organ and tumor(s).

It will therefore be appreciated that each of the radioembolic agents described herein can include a gamma emitter radioisotope to provide the diagnostic tool discussed above and more particularly, to allow for gamma-emission imaging to confirm the radioactivity and the extend of penetration of the embolized tumor and be used for dosimetry calculations.

### Microcatheters

Microcatheters, including neuromicrocatheters, are generally microtubes inserted into the body through a blood vessel such as the femoral artery and have a variety of uses. A syringe is typically used in combination with the microcatheter to inject the liquid into the microcatheter. Microcatheters have a distal and a proximal end where, typically, at or near the very distal end, a marker band can be employed to permit the clinician to visualize the microcatheter positioning during in vivo use. The marker band typically comprises a metal or metal alloy ring such as platinum, nitinol and/or gold rings which can be visualized via fluoroscopy to allow the user to easily ascertain the location of the distal tip.

Microcatheters are typically used to embolize the neurovasculature in a relatively non-invasive manner. A variety of microcatheters, suitable for the wide variety of applications, are available commercially and are suitable for use herein.

In accordance with the present disclosure, the microcatheter (delivery catheter) is configured for use in the CNS concurrently with delivery of a liquid radioembolic agent as described herein.

Prior to the procedure, multiple syringes (e.g., 3 cc syringes) can be prepared with an aqueous solution, such as D5 water (dextrose 5% in water). One or more syringes (e.g., a 3 cc syringe) with the radioembolic agent is also prepared. The microcatheter is flushed with the aqueous solution (e.g., D5 water) to clear the microcatheter of any blood product prior to delivery of the radioembolic agent through the microcatheter.

The liquid radioembolic agent is delivered with the microcatheter under live imaging (e.g., live fluoroscopy) to the target embolization location. The delivery is such that the radioembolic agent embolizes deep into the tumor vessels resulting in deep penetration. Once embolic material appears on the imaging display, the forward pressure on the syringe is adjusted to embolize more distally verses more proximally, while monitoring for signs of reflux. Once the user notices that there is no forward flow of the embolic material, the microcatheter should be immediately withdrawn.

Once the user is satisfied with the degree of penetration and the amount of embolization, aspirate on the microcatheter and associated syringe to remove these devices. Post-procedure imaging, such as an angiogram, is performed to evaluate the tumor embolization (See, figures).

### Dosimetry

As is known, dosimetry is the determination and measurement of the amount or dosage of radiation absorbed by a substance or living organism by means of a dosimeter. Dosimetry can therefore be defined as the amount of absorbed dose delivered by ionizing radiation. Absorbed dose is the fundamental quantity defined as the mean energy imparted by the radiation per unit mass. The common SI unit for radioactivity is the becquerel (Bq), which is commonly measured in gigabequerel (GBq) in certain applications, but also referenced as millicurie (mCi). The activity (GBq or mCi) when deposited into a specific volume of specific tissue results in a distribution of energy, referred to as dose (Gy).

### Image Based Dosimetry

Image based dosimetry uses images, e.g., MRI, SPECT/CT, PET/CT, etc.) to calculate a more accurate dose based on pre-treatment or post-treatment images. While consistently investigated, image based dosimetry recommendations have yet to solidify a standardized consensus on the methodology of calculating image-based doses. Compared to the current clinical dosimetric methods, image based dosimetry relies more directly on images obtained from the pre-treatment or post-treatment imaging steps. Independent of tumor burden, tumor segmentation, or tumor uptake fractions, image based dosimetry estimations rely mainly on image quality such as image resolution and reconstruction parameters. Moreover, image based dosimetry makes fewer assumptions than the current clinical dosimetric methods.

After the therapeutic treatment is completed, post-treatment image based dosimetry may be used to retrospectively quantify the absorbed dose of an administered treatment.

Volumetric imaging (MRI) can be used to quantify extent of tumor embolization and associated tissue necrosis by comparing pre-embolization to post-embolization imaging. Loss of contrast enhancement corresponds to tumor embolization: Areas of tumor perfused on pre-embolization T1+C MRI sequences [contrast enhancement appears as white] but not perfused on post-embolization T1+C [loss of contrast enhancement appears as dark/black] have been embolized. Based on the foregoing, dosimetry calculations can be made.

Homogeneity Permitting Precise Dosimetry: As a result of the homogenous makeup of the liquid radioembolic agent, it is possible to deliver uniform and predictable levels of radioactivity that can be precomputed as part of the patient care algorithm. Total dose to tumor can be computed, and total dose post-treatment to tumor and periphery can be computed, whatever radioisotope is used. More specifically, the use of gamma radiation allows for confirmatory imaging and precise computation of dosimetry in delivering the liquid embolics (to perform liquid brachytherapy). The present disclosure teaches a system and method that combine a homogeneously active agent, deep penetration into tissue, and the ability (via gamma emission) to image (including during therapy in real-time) the distribution of radiation within the targeted (tumor) tissue. This enables precise dosimetric calculations in ways that are not possible with conventional particle radioembolization and in ways that are neither disclosed nor contemplated by conventional systems and methods. As previously mentioned, the gamma camera (FIG. 7) shows homogeneous distribution of radiation around a radio-embolus.

In yet another aspect, the successful treatment of tumors using the radioembolic system and embolization techniques described herein was confirmed by performing volumetric analysis of embolized tumors. The volumetric analysis of tumor necrosis provides indication of therapeutic efficacy and therefore are many different volumetric analysis software modules that can be used to perform the volumetric analysis. For example, a direct assessment of the extent of embolization through volumetric segmentation on pre- and post-embolization images (e.g., magnetic resonance imaging (MRI)). For example, in one study in treating a meningioma using the radioembolic agents described herein, the vascular delivery pathway was through the middle meningeal artery and volumetric analysis of the embolized tumor revealed a percent embolization of 91.28%. In other studies, for different tumor locations, the percent embolization ranged from 31.30% to 59.34% which was indicative of successful embolization at the target site. In addition, gamma camera imaging (discussed herein) confirmed effective radiation treatment of the tumor (target tissue).

### Diverse Therapeutic Applications

As discussed herein, the liquid radioembolic agents described herein comprise radiopharmaceuticals that can be used in many different therapeutic applications, including but not limited to vascular, interstitial or topical applications. Accordingly, the present liquid radioembolics can be used to treat benign or malignant tumors of the breast, prostate, lung, colon, liver, pancreas, skin, head and neck, brain, etc. For example, the present teachings can be applied to skin tumors (and melanomas of various types), lung tumors, colon and intestinal tumors, breast tumors, prostate tumors, lymphoma, renal tumors, endometrial tumors, liver tumors, pancreatic tumors, bladder tumors, thyroid tumors, bone tumors (including myeloma and other hematologic processes), head and neck tumors, brain tumors (benign, such as meningioma, and malignant, such as metastasis or glioma). Additionally, other pathologic entities in all of these locations that may respond to radiation therapy can potentially be treated by use of the disclosed radioembolics and related embolization treatments. Additionally, applications for the disclosed radioembolics include the treatment of arteriovenous malformations (AVM), other vascular malformations, “other” hyperemic tissues, etc. Thus, the present teachings can be applied to treatment of vascular malformations and are not limited to the treatment of tumors.

It will therefore be appreciated that while the present disclosure includes examples directed to CNS applications, such teachings and examples are only exemplary and not limiting of the teachings of the present disclosure.

The invention encompassed by the present disclosure has been described with reference to the accompanying drawings, which form a part hereof, and which show, by way of illustration, example implementations and/or embodiments. As such, the figures and examples above are not meant to limit the scope of the present application to a single implementation, as other implementations are possible by way of interchange of some or all of the described or illustrated elements, without departing from the spirit of the present disclosure. Among other things, for example, the disclosed subject matter can be embodied as methods, devices, components, or systems.

Moreover, where certain elements of the present application can be partially or fully implemented using known components, only those portions of such known components that are necessary for an understanding of the present application are described, and detailed descriptions of other portions of such known components are omitted so as not to obscure the application. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present application encompasses present and future known equivalents to the known components referred to herein by way of illustration.

Furthermore, it is recognized that terms used herein can have nuanced meanings that are suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter can be based upon combinations of individual example embodiments, or combinations of parts of individual example embodiments.

The foregoing description of the specific implementations will so fully reveal the general nature of the application that others can, by applying knowledge within the skill of the relevant art(s) (including the contents of the documents cited and incorporated by reference herein), readily modify and/or adapt for various applications such specific implementations, without undue experimentation, without departing from the general concept of the present application. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown of drawings are shown accordingly to one example and other dimensions can be used without departing from the present disclosure.

While various implementations of the present application have been described above, it should be understood that they have been presented by way of example, and not limitation. It would be apparent to one skilled in the relevant art(s) that various changes in form and detail could be made therein without departing from the spirit and scope of the disclosure. Thus, the present disclosure should not be limited by any of the above-described example implementations, and the invention is to be understood as being defined by the recitations in the claims which follow and structural and functional equivalents of the features and steps in those recitations.

What is claimed is:

1. A liquid radioembolic agent for delivering local radiation to a target location while also causing devascularization at the target location, the liquid radioembolic agent comprising:
a biocompatible prepolymer;
a first radioisotope having a first type of ionizing radiation for treatment of a first region of the target location; and
a second radioisotope having a second type of ionizing radiation for treatment of a second region of the target location, the second type of ionizing radiation being different than the first type of ionizing radiation;
wherein each of the first type of ionizing radiation and the second type of ionizing radiation is selected from the group consisting of: alpha type ionizing radiation, beta type ionizing radiation, gamma type ionizing radiation, and combinations thereof.

2. The liquid radioembolic agent of claim 1, wherein the liquid embolic agent comprises a homogenous liquid mixture.

3. The liquid radioembolic agent of claim 1, wherein the target location comprises a tumor and the first type of ionizing radiation comprises beta type ionizing radiation and the first region of the tumor comprises a core of the tumor and the second type of ionizing radiation comprises alpha type ionizing radiation and the second region of the tumor comprises a periphery of the tumor.

4. The liquid radioembolic agent of claim 1, wherein the target location comprises a tumor and the first type of ionizing radiation comprises alpha type ionizing radiation and the first region of the tumor comprises a core of the tumor and the second type of ionizing radiation comprises beta type ionizing radiation and the second region of the tumor comprises a periphery of the tumor.

5. The liquid radioembolic agent of claim 4, wherein one of the first radioisotope and the second radioisotope comprises Iodine-131 which is both a beta and gamma type emitter.

6. The liquid radioembolic agent of claim 1, wherein the biocompatible prepolymer comprises a cyanoacrylate.

7. The liquid radioembolic agent of claim 1, wherein one of the first radioisotope and the second radioisotope comprises one of Iodine-125 and Iodine-131.

8. The liquid radioembolic agent of claim 1, wherein the target location comprises target tissue or a vascular malformation.

9. An embolization kit for delivering local radiation to a tumor while also causing tumor devascularization due to embolization, the kit comprising:
a first liquid radioembolic agent for treatment of at least a first region of the tumor, the first liquid radioembolic agent comprising:
a biocompatible polymer or prepolymer;
a first radioisotope having a first type of ionizing radiation for treatment of the first region of the tumor; and
a second liquid radioembolic agent for treatment of at least a second region of the tumor, the second liquid radioembolic agent comprising:
a biocompatible polymer or prepolymer;
a second radioisotope having a second type of ionizing radiation for treatment of the second region of the tumor, the second type of ionizing radiation being different than the first type of ionizing radiation;
wherein each of the first type of ionizing radiation and the second type of ionizing radiation is selected from the group consisting of: alpha type ionizing radiation, beta type ionizing radiation; gamma type ionizing radiation; and combinations thereof.

10. The kit of claim 9, wherein the first liquid radioembolic agent is loaded within a first microcatheter and the second liquid radioembolic agent is loaded within a second microcatheter.

11. The kit of claim 9, wherein the first region of the tumor is the center and the second region of the tumor is the periphery.

12. The kit of claim 9, wherein each of the first liquid radioembolic agent and the second liquid radioembolic agent comprises a homogenous liquid mixture.

13. The kit of claim 9, wherein the first type of ionizing radiation comprises beta type ionizing radiation and the first region of the tumor comprises a core of the tumor and the second type of ionizing radiation comprises alpha type ionizing radiation and the second region of the tumor comprises a periphery of the tumor.

14. The kit of claim 9, wherein the first type of ionizing radiation comprises alpha type ionizing radiation and the first region of the tumor comprises a core of the tumor and the second type of ionizing radiation comprises beta type ionizing radiation and the second region of the tumor comprises a periphery of the tumor.

15. The kit of claim 9, wherein one of the first radioisotope and the second radioisotope comprises Iodine-131 which is both a beta and gamma type emitter.

16. The kit of claim 9, wherein the biocompatible prepolymer of each of the first liquid radioembolic agent and the second liquid radioembolic agent comprises a cyanoacrylate.

17. The kit of claim 9, wherein one of the first radioisotope and the second radioisotope comprises one of Iodine-125 and Iodine-131.

18. The kit of claim 11, wherein the first radioisotope comprises a beta emitter radioisotope selected from the group consisting of: I-125, I-131, Y-90, Lu-177, and Cu-67 and the second radioisotope comprises an alpha emitter radioisotope selected from the group consisting of: Pb-212, Ac-225, Ra-223, and At-211.

19. The kit of claim 9, wherein at least one of the first radioisotope and the second radioisotope comprises from about 0.1 to about 25 weight percent of the respective first liquid radioembolic agent or the second liquid radioembolic agent and has a radioactive content of from about 0.5 microcurie to about 100 millicurie.

20. The kit of claim 9, wherein the tumor comprises a meningioma.

21. A method for embolizing a blood vessel leading to or in a solid mass tumor and causing necrosis to a portion of the solid mass tumor, the method comprising the steps of:
identifying at least one blood vessel that leads to or is in the solid mass tumor;
injecting a first liquid radioembolic agent into the at least one blood vessel, the first liquid radioembolic agent including a biocompatible polymer or prepolymer and a first radioisotope and being injected into the blood vessel under conditions wherein the polymer or prepolymer polymerizes and forms a solid mass which embolizes the at least one blood vessel and further wherein the first radioisotope is employed in an amount effective to cause necrosis of at least a first portion of the tumor; and
injecting a second liquid radioembolic agent into the at least one blood vessel, the second liquid radioembolic agent including a biocompatible polymer or prepolymer and a second radioisotope and being injected into the blood vessel under conditions wherein the polymer or prepolymer polymerizes and forms a solid mass which embolizes the at least one blood vessel and further wherein the second radioisotope is employed in an amount effective to cause necrosis of at least a second portion of the tumor.

22. The method of claim 21, wherein the at least one blood vessel includes at least one first vascular pedicle that leads to the first portion of the tumor and at least one second vascular pedicle that leads to the second portion of the tumor, the first liquid radioembolic agent being injected into the first vascular pedicle and the second liquid radioembolic agent being injected into the second vascular pedicle.

23. The method of claim 22, wherein the first portion of the tumor comprises a center of the tumor and the second portion of the tumor comprises a periphery of the tumor.

24. The method of claim 22, wherein the first radioisotope is different than the second radioisotope.

25. The method of claim 21, wherein the first radioisotope has a first type of ionizing radiation and the second radioisotope has a second type of ionizing radiation, wherein each of the first type of ionizing radiation and the second type of ionizing radiation is selected from the group consisting of: alpha type ionizing radiation, beta type ionizing radiation, gamma type ionizing radiation, and combinations thereof.

26. The method of claim 25, wherein the first type of ionizing radiation comprises beta type ionizing radiation and the first portion of the tumor comprises a core of the tumor and the second type of ionizing radiation comprises alpha type ionizing radiation and the second portion of the tumor comprises a periphery of the tumor.

27. The method of claim 25, wherein the first type of ionizing radiation comprises alpha type ionizing radiation and the first portion of the tumor comprises a core of the tumor and the second type of ionizing radiation comprises beta type ionizing radiation and the second portion of the tumor comprises a periphery of the tumor.

28. The method of claim 21, wherein one of the first radioisotope and the second radioisotope comprises a beta type emitter and the other of the first radioisotope and the second radioisotope comprises an alpha type emitter.

29. The method of claim 21, wherein the biocompatible prepolymer of each of the first liquid radioembolic agent and the second liquid radioembolic agent comprises cyanoacrylate.

30. The method of claim 21, wherein the first radioisotope comprises one of a beta emitter radioisotope selected from the group consisting of: I-125, I-131, Y-90, Lu-177, and Cu-67 and an alpha emitter radioisotope selected from the group consisting of: Pb-212, Ac-225, Ra-223, and At-211 and the second radioisotope comprises one of a beta emitter radioisotope selected from the group consisting of: I-125, I-131, Y-90, Lu-177, and Cu-67 and an alpha emitter radioisotope selected from the group consisting of: Pb-212, Ac-225, Ra-223, and At-211.

31. A method for treatment of a target tissue comprising the steps of:
identifying at least one first blood vessel that leads to or is in the target tissue;
injecting a first liquid radioembolic agent into the at least one blood vessel, the first liquid radioembolic agent including a biocompatible polymer or prepolymer and a first radioisotope and being injected into the at least one first blood vessel under conditions wherein the polymer or prepolymer polymerizes and forms a solid mass which embolizes the at least one first blood vessel and further wherein the first radioisotope is employed in an amount effective to cause necrosis of at least a first portion of the target tissue; and
injecting a second liquid radioembolic agent into the at least one first blood vessel or into at least one second blood vessel, the second liquid radioembolic agent including a biocompatible polymer or prepolymer and a second radioisotope and being injected into the at least one first blood vessel or the at least one second blood vessel under conditions wherein the polymer or prepolymer polymerizes and forms a solid mass which embolizes the at least one first blood vessel or the at least one second blood vessel and further wherein the second radioisotope is employed in an amount effective to cause necrosis of at least a second portion of the target tissue.

32. The method of claim 31, wherein the tissue comprises one of a solid mass tumor and a vascular malformation.

33. The method of claim 32, wherein the tumor comprises a meningioma.

34. The method of claim 30, wherein at least one of the first liquid radioembolic agent and the second liquid radioembolic agent includes a gamma emitter radioisotope for performing subsequent confirmatory imaging and precise computation of dosimetry.

35. The method of claim 34, further including the step of using a gamma camera in real time to image a distribution of radiation within the target tissue.

36. The method of claim 35, further including the step of obtaining a plurality of gamma camera images over a predetermined period of time to determine and observe radiation levels over the predetermined period of time and using the plurality of gamma camera images to perform dosimetric calculations.
